(19) United States
(12) Patent Application Publication
(10) Pub. No.: US 2019/0346463 A1
(43) Pub. Date: Nov. 14, 2019

(54) COMPOSITIONS AND METHOD FOR DETERMINING INSULIN RESISTANCE USING NON-ESTERIFIED FATTY ACID ANALYSIS

(71) Applicant: The Penn State Research Foundation, University Park, PA (US)

(72) Inventors: Gregory SHEARER, State College, PA (US); Rachel WALKER, State College, PA (US); Jennifer FORD, State College, PA (US); Michael GREEN, State College, PA (US)

(21) Appl. No.: 16/347,569

(22) PCT Filed: Nov. 6, 2017
(86) PCT No.: PCT/US17/60096

§ 371 Date: May 3, 2019

Related U.S. Application Data
(60) Provisional application No. 62/417,681, filed on Nov. 4, 2016.

(51) Int. Cl. G01N 33/92

(52) U.S. Cl. CPC G01N 33/92; G01N 2800/042

(57) ABSTRACT

Provided are methods for assessing samples to determine or to aid in determining Insulin Resistance and/or malfunctioning adipocytes. The methods involve analysis of non-esterified fatty acids (NEFAs) trafficking by determining kinetic parameters that include the fractional rate of NEFA appearance (Ra), the rate of NEFA disappearance (kNEFA), threshold for glucose-induced suppression (tG), and IC50 for glucose inhibition of NEFA production (glCso). Determining Ra for stearate that is similar to a control value, and/or a kNEFA for stearate that is higher than a control, indicates that the individual from whom the sample was taken has IR and/or adipocyte dysfunction. The method can include analysis of trafficking patterns for one or more of myristic, palmitic, palmitoleic, oleic, and linoleic acids, in addition to stearate. A determination of IR and/or adipocyte dysfunction can indicate that the individual is a candidate for treatment with an agonist of a peroxisome proliferator-activated receptor.

### CROSS REFERENCE TO RELATED APPLICATIONS

This application claims priority to U.S. provisional application No. 62/417,681, filed Nov. 4, 2016, the disclosure of which is incorporated herein by reference.

### STATEMENT REGARDING FEDERALLY SPONSORED RESEARCH

This invention was made with government support under Grant No. 2011-67001-30117, awarded by the United States Department of Agriculture. The Government has certain rights in the invention.

### FIELD

The present disclosure relates generally to metabolic disorders and more particularly to adipocyte involvement in insulin resistance.

### BACKGROUND

Current tests for determining insulin resistant glycemic indicators of insulin resistance primarily rely on analysis of glucose metabolism alone. Furthermore, diagnosis of insulin resistance does not occur until an individual is already experiencing symptoms and pathology. Thus, there is an ongoing and unmet need for an improved test that could identify insulin resistance before pathology emerges and does not rely on glucose metabolism alone. The present disclosure is pertinent to this and other needs

### SUMMARY

Embodiments of this disclosure relate to adipocyte insulin resistance (IR) and its relationship to metabolism of non-esterified fatty acids (NEFAs). In particular, the disclosure in part reveals novel relationships between NEFA release and adipocyte insulin resistance (IR).

In embodiments the disclosure provides methods for determining IR and/or adipocyte dysfunction. In one approach a method comprises analyzing a sample(s) from an individual to whom a glucose test is administered. The analysis comprises evaluation of one or more NEFA trafficking parameters. The trafficking parameter are selected from: a fractional rate of NEFA appearance (Rₐ), a rate of NEFA disappearance (kNEFA), a threshold for glucose-induced suppression (tG), and IC₅₀ for glucose inhibition of NEFA production (gIC₅₀). The analysis includes at least evaluation of trafficking for stearate, which as is well known in the art is an example of a NEFA. Performance of the method enables a determination that the individual from whom the sample was taken has IR and/or adipocyte dysfunction. This determination can be made by, for example, determining that the Rₐ for stearate is approximately the same as a suitable control, and/or that the kNEFA for stearate is higher than a suitable control. In embodiments ratios of one or more NEFA trafficking parameters can be determined, and can be compared to suitable controls.

In certain non-limiting implementations, in addition to stearate, the sample is analyzed for the trafficking parameters for one or more of myristic, palmitic, palmitoleic, oleic, and linoleic acid. In certain non-limiting examples, the control for the Rₐ for stearate and/or the control for the kNEFA for stearate is an Rₐ or a kNEFA, respectively, for at least one of myristic, palmitic, palmitoleic, oleic, and linoleic acids. In a non-limiting implementation, a suitable control comprises NEFA trafficking parameters from a sample from the individual that is obtained before the glucose challenge, and a test sample is obtained from the individual subsequent to the glucose challenge.

In certain embodiments, an individual is identified using a method of this disclosure to be a candidate for treatment with an agonist of a peroxisome proliferator-activated receptor (PPAR). In embodiments the PPAR is administered to the individual. The efficacy of the PPAR inhibitor can be assessed using methods of this disclosure, such as by repeating a test of the invention one or more additional times during a treatment period.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The present disclosure relates in certain aspects to metabolism of non-esterified fatty acids (NEFAs). In particular, as known in the art insulin suppresses intracellular lipolysis in adipocytes, and poor suppression of non-esterified fatty acid (NEFA) release is directly related to adipocyte insulin resistance (IR). Prior to the present disclosure it was not known whether the insulin-mediated suppression is uniform for all fatty acids (FAs). In this regard, the present disclosure is believed to reveal for the first time that NEFAs are differently trafficked by adipocytes in insulin resistant subjects, relative to NEFA trafficking in healthy control individuals. Thus, and without intending to be bound by any particular theory, the present disclosure provides methods for IR diagnosis by analysis of NEFA trafficking at several time points, or at a single time point, to identify IR individuals earlier than currently available tests.

In general the present disclosure involves analysis of NEFA trafficking by determining kinetic parameters that include, but are not necessarily limited to, the fractional rate of NEFA appearance (Rₐ), the rate of NEFA disappearance (kNEFA), threshold for glucose-induced suppression (tG), and IC₅₀ for glucose inhibition of NEFA production (gIC₅₀). Those skilled in the art will recognize that each of these parameters is known and can be determined using a variety of established approaches. In this regard the presence or absence, qualitative and quantitative amounts of, and kinetic parameters for one or more NEFAs in a biological sample can be sample determined using, for example, chromatographic methods, such as column chromatography, including High Performance Liquid Chromatography (HPLC) and gas chromatography, and spectroscopy methods including mass spectrometry, nuclear magnetic resonance spectroscopy, infrared spectroscopy, positron emission tomography and other approaches, such as those described in “A novel minimal model to describe NEFA kinetics following an intravenous glucose challenge” Ray C. Boston, Peter J. Moate, American Journal of Physiology—Regulatory, Integrative and Comparative Physiology Apr 2008, 294 (4) R1140-R1147.

In certain embodiments the disclosure comprises determining kinetic parameters for one or a combination of myristic, palmitic, palmitoleic, stearic, oleic, and linoleic acids. Thus, any 1, 2, 3, 4, 5, or 6 of these NEFAs can be analyzed. In embodiments, kinetic parameters are determined for a combination of these NEFAs, wherein the combination includes kinetic parameters for at least stearate trafficking. Thus, parameters for stearate can be determined in conjunction with parameters for any combination or all of myristic, palmitic, palmitoleic, oleic, and linoleic acids. The chemical composition and structure of each of these NEFAs is well known in the art. In certain implementations the disclosure comprises determining trafficking of one or more NEFAs that is different from a control, wherein determining such a difference is indicative of IR, or comprises a diagnosis of IR, or aids in a physician's diagnosis of IR. In an embodiment a difference in trafficking of stearate relative to another NEFA in the same sample, or a difference in trafficking of stearate relative to a suitable control is indicative of IR. In addition to or as an alternative to indicia of IR, differential trafficking of NEFAs, such as stearate, can comprise an indication that the individual from whom the sample was obtained has dysregulated adipocytes, such as adipocytes that exhibit reduced or altered suppression of release of one or more NEFAs, such as in response to a glucose challenge as described further below.

In one aspect the disclosure comprises analyzing a sample from an individual and determining at least one of Rₐ, kNEFA, tG, and IC₅₀ and gIC₅₀ for one or more NEFAs. Analysis of all single and all combinations of parameters and all single and all combinations of NEFAs are encompassed by this disclosure. In one approach the disclosure comprises a method for determining IR and/or adipocyte dysfunction by analyzing at least a first sample from an individual to whom a glucose challenge is administered for a NEFA parameter, wherein a difference in one or more of the trafficking parameters relative to a control is indicative that the individual has the IR and/or the adipocyte dysfunction. In embodiments ratios of one or more NEFA trafficking parameters can be determined, and can be compared to suitable controls.

In one approach the disclosure comprises determining at least one of the parameters for at least stearate and at least one other NEFA. In one embodiment the method comprises determining at least Rₐ or kNEFA for stearate and one other NEFA. In an embodiment the method comprises determining Rₐ and kNEFA for stearate and at least one other NEFA. In an embodiment the method comprises determining at least Rₐ and kNEFA for stearate and 3, 4, or 5 other NEFAs. In an embodiment the method comprises determining at least Rₐ and kNEFA for all six NEFAs described herein. In one embodiment, the disclosure comprises determining at least Rₐ, kNEFA for stearate, and at least one other of tG, and IC₅₀ and gIC₅₀ for one or more NEFAs other that stearate. In an embodiment the disclosure comprises determining all of Rₐ, kNEFA, tG, and IC₅₀ and gIC₅₀ for all six NEFAs described herein. The method in various implementations comprises comparing Rₐ for at least one non-stearate NEFA with Rₐ for stearate, wherein if the Rₐ for stearate is approximately the same as a suitable control, but the Rₐ for the at least one non-stearate NEFA is lower than the control, it is indicative that the individual from whom the sample was taken has IR. In another embodiment, the method comprises comparing kNEFA for at least one non-stearate NEFA with kNEFA for stearate, wherein if the kNEFA for stearate is higher than a suitable control, but the kNEFA for the at least one non-stearate NEFA is about the same than the control, it is indicative that the individual from whom the sample was taken has IR. Notwithstanding the foregoing, controls used with the present disclosure can comprise any suitable reference, including but not limited to a standardized value, an area under a curve, a value taken before a glucose challenge, a value obtained from testing samples from individuals who do not have IR, or who are known to have. In certain approaches the value determined for stearate is compared to the same value obtained for one or a combination of the other NEFAs. In certain embodiments the value represents at least two measurements, wherein one measurement is taken before a glucose challenge, a second measurement is taken after a glucose challenge, but it is expected that the method may be performed using a single sample taken after a glucose challenge.

The invention is generally suitable for use with any biological sample obtained from an individual, but will generally be performed using blood. Any suitable techniques can be used to obtain the biological sample. The biological sample can be tested directly, or it can be subjected to a processing step before testing.

As noted above, the disclosure in certain embodiments comprises testing a biological sample from an individual who has been administered glucose as part of a glucose tolerance test procedure, or any other type of glucose challenge. The disclosure includes taking a baseline sample from an individual before a glucose test and testing the sample for one or any combination of the NEFA trafficking parameters described herein, and subsequently obtaining and testing a second sample at one or more time points after the glucose challenge. The time point at which the sample is obtained after the glucose challenge can range, but in certain embodiments will be from about 30-90 minutes, or 40-70 minutes. In general, the steps and material components involved in administration of glucose tolerance tests and analysis of metabolite levels (other than as described herein for NEFA trafficking) are well known in the art and are part of routine medical examinations for a wide variety of individuals, and any type of glucose test can be used for this invention, including . Briefly, a standard amount of glucose is given to the individual and blood sample(s) taken subsequently are analyzed for parameters related to clearance of glucose from the blood. The glucose can be administered orally or intravenously. In embodiments, an initial fasting plasma glucose level can be determined before the glucose is administered, and the fasting glucose level can be compared to standard controls and/or to the glucose levels after a period time subsequent to administration of the glucose. The period of time after the glucose administration can range but is generally a period of between one and four hours. In certain aspects an oral glucose challenge test (OGCT) can be performed such that no fasting is required, and the results can be interpreted within about one hour; the OGCT is typically used to check pregnant women for gestational diabetes. In certain aspects a sample from an individual who has had a glucose administration pursuant to a frequently sampled intravenous glucose tolerance test (FSIVGTT) can be used. As is known in the art, a FSIVGTT is a test that measures response to a high dose of intravenous (IV) glucose and insulin. It is generally administered to an individual who has fasted prior to the administration. Generally, a first blood sample is taken at 0 minutes, after which glucose is administered at a constant rate over about a 2 minute period in an amount in proportion to the body weight of the individual. After a period of time, generally about twenty minutes after the start of the test, insulin is administered in a dosage also determined by body weight, and blood samples are frequently taken over what is typically a four-hour period. Thus, the samples analyzed in embodiments of the present disclosure may be obtained from an individual who has been subjected to any of a variety of tests that involve administration of glucose, and may also involve administration of insulin. In certain examples, an individual who is tested according to a method of this disclosure is determined by way of a glucose tolerance test to not have a metabolic disorder such as IR, but is nevertheless determined to have IR and/or dysfunctional adipocytes due to a result of the present test.

In certain embodiments, the sample is obtained from an individual who has been diagnosed with or is at risk for developing any form of a metabolic disorder and/or is clinically obese. In embodiments, a sample tested in the method of the present disclosure is obtained from an individual who has been previously determined to have a metabolic disorder. In embodiments, the sample may be obtained from an individual who is undergoing treatment for a metabolic disorder. Thus, multiple measurements can be obtained prior to, during, and/or subsequent to a metabolic disorder treatment or other intervention, including but not necessarily limited to dietary changes and/or implementation or alterations in an exercise program. Serial measurements can be taken monitor the progression of the disorder and/or the efficacy of any particular approach to treating the metabolic disorder.

In embodiments, the disclosure comprises determining that the individual has IR and/or a condition that affects adipocytes. Thus, in various embodiments the disclosure may identify individuals who have or are at risk for developing other metabolic disorders that may be associated with IR, including but not necessarily limited to dyslipidemia, diabetes and metabolic syndrome, or combined lipid and glucose dysregulation, and/or morphological indicators of adipocyte dysregulation, such as enlargement of adipocyte cell size.

The disclosure comprises in certain approaches identification of the presence (or absence) of dysregulated adipocytes in an individual, or in a biological sample, by determining NEFA kinetic parameters as described herein. In this regard, the present disclosure may be pertinent to either or both types of adipocytes commonly known as white fat cells (also referred to monovacuolar cells) and brown fat cells (also referred to as plurivacuolar cells). Adipocytes in brown fat differ from white adipocytes in various properties, including morphological features and in that the brown fat adipocytes express uncoupling protein-1 (UCP-1). In certain non-limiting examples dysregulated adipocytes may also exhibit gene expression profiles that are distinct from healthy adipocytes, such as changes in expression of genes related to either fatty acid storage (i.e., acyl CoA synthase (ACSL1), hormone-sensitive lipase (LIPE), aquaporin 7 (AQP7), perilipin (PLIN) or cell adhesion (fibronectin FN1, collagens COL1A1, COL1A3, metalloprotein MMP9, Lipoprotein Lipase (LIPD), stearyl-CoA desaturase (SCD), fatty acid desaturases (FADS2 or FADS2), fatty acid elongases (ELOVL3, 5 or 6), or both (i.e., scavenger receptor FAT/CD36).

In certain approaches the disclosure comprises testing a sample, determining that the individual from whom the sample was obtained has or is at risk for developing a metabolic disorder, such as IR, where after the individual is prescribed a change in diet, an initiation of or modification of an exercise regimen, or is prescribed (and administered) a therapeutic agent. The therapeutic agent can be any agent that is known to or determined to affect NEFA trafficking by adipocytes. In certain approaches the therapeutic agent is a target of cell surface receptor that is expressed by adipocytes, or the target is a nuclear receptor protein. In this regard, the peroxisome proliferator-activated receptors (PPARs) are a group of nuclear receptor proteins that function as transcription factors regulating the expression of genes. All PPARs are known to heterodimerize with the retinoid X receptor (RXR) and bind to specific regions on the DNA of target genes called peroxisome proliferator hormone response elements (PPREs). PPARs play essential roles in the regulation in a variety of pathways and metabolism of carbohydrates, lipids, and proteins. In certain approaches the disclosure comprises testing a sample and determining that NEFAs in the sample were produced by dysfunctional adipocytes.

The PPAR family includes PPAR-α, PPAR-γ, and PPAR-δ, the latter sometimes referred to as PPAR-β. PPAR-α is expressed in liver, kidney, heart, muscle, adipose tissue, as well as other tissues. PPAR-δ is expressed in many tissues but markedly in brain, adipose tissue, and skin. PPAR-γ includes three splice variants that have distinct expression patterns. PPAR-γ is expressed in most tissues, while PPAR-γ2 is expressed mainly in adipose tissue. PPAR-γ3 is expressed in macrophages, large intestine, and white adipose tissue. PPAR agonists are used in treating diabetes mellitus and other diseases that feature insulin resistance. Thus, the disclosure includes in various embodiments identification of an individual who is a candidate for therapy with a PPAR-agonist. The disclosure further includes prescribing and/or administering to the PPAR agonist to the individual. Thus, in certain aspects, an individual diagnosed according to an embodiment of this disclosure is prescribed and/or is administered a PPAR agonist. In embodiments the PPAR agonist is a PPAR pan-agonist. In embodiments, the PPAR agonist comprises or consists of a compound capable of stimulating one or two of the known PPAR signaling pathways. Representative such compounds include selective PPAR agonists. Non-limiting examples of PPAR agonists expected to be suitable with aspects of this discourse include taleglitazar, farglitazar, muraglitazar, tesaglitazar, and any thiazolidinedione (TZD). Exemplary TZDs include pioglitazone (ACTOS), rosiglitazone (AVANDIA), rivoglitazone, and troglitazone. Several TZDs, including troglitazone, rosiglitazone, and pioglitazone, have insulin-sensitizing and anti-diabetic activity in humans with type 2 diabetes and impaired glucose tolerance. Farglitazar is a very potent non-TZD PPAR-γ -selective agonist that exhibits antidiabetic as well as lipid-altering activity in humans. The PPAR agonist can be administered to the individual and methods of this disclosure can be used to monitor the efficacy of the therapy, such as by periodic blood tests to determine whether or not NEFA trafficking in the individual is normalizing, i.e., the NEFA trafficking is or is trending toward a NEFA trafficking profile of an individual who does not have IR. The dosage a PPAR agonist will depend on the disease state and other clinical factors, such as weight and condition of the subject, the subject's response to the therapy, the type of formulations and the route of administration. The precise dosage to be therapeutically effective and non-detrimental can be determined by those skilled in the art. PPAR agonists can be administered using any suitable route, including but not necessarily limited to oral, intravenous and subcutaneous routes. In addition to, or as an alternative, other agents and treatment modalities can be implemented, including but not necessarily limited to surgical interventions to produce and/or promote weight loss, such as liposuction, Roux-en-Y gastric bypass, laparoscopic adjustable gastric banding, sleeve gastrectomy, and duodenal switch with biliopancreatic diversion. Other pharmaceutical approaches can comprise administration of insulin, including short, intermediate and rapid acting formulations, amylinomimetic drugs, alpha-glucoside inhibitors, biguanides such as metformin, dopamine agonists, DPP-4 inhibitors, incretin mimetics, meglitinides, sodium glucose transporter 2 (SGLT2) inhibitors, sulfonylureas, non-steroidal anti-inflammatory drugs (NSAIDs), any fat absorption inhibitor, any anti-hyperlipidemic agent, any agent that promotes remodeling of skeletal muscle to an oxidative phenotype.

The following specific example is provided to illustrate the invention, but is not intended to be limiting in any way.

### EXAMPLE 1

We used a compartmental model of NEFA trafficking in response to a glucose challenge. The present Example provides an analysis of 1) a determination if trafficking of all FAs is uniform, and 2) determine whether the trafficking is altered in IR.

This Example reflects an ancillary study. In order to obtain the results described below, optimally healthy (n=15) and IR (n=60) subjects with Homeostatic Model Assessment for Insulin Resistance (HOMA) scores of 0.65 [0.59, 0.88] and 3.21 [2.34, 4.36] respectively (p<0.0001 by X²) were given a frequently sampled intravenous glucose tolerance test. Determination and interpretation of HOMA scores are well within the purview of those skilled in the art, and tools for calculating HOMA scores are publicly available. Four subjects from each group were selected by stratifying IR. Six FAs were measured by GC-MS (myristic, palmitic, palmitoleic, stearic, oleic, and linoleic acids). Kinetic parameters representing the fractional rate of NEFA appearance (Rₐ), the rate of NEFA disappearance (kNEFA), threshold for glucose-induced suppression (tG) and IC₅₀ for glucose inhibition of NEFA production (gIC₅₀) were estimated. Differences are reported as mean [95% CI].

All data is reported as Mean [95% CI]. Using the foregoing materials and methods we determined Rₐ was highest for linoleate (10.6 [7.8,14.5]%/min) and lowest for stearate (2.7 [2.0, 3.7]%/min). Rₐ was 48% [24, 64] lower in IR (p=0.02) for all FAs except stearate, which was unchanged. Mean kNEFA (%/min) for the unsaturated FAs was 106% [50, 183] higher than for palmitate. In IR, only kNEFA for stearate was greater, by 55% [14, 76] (p=0.05). The tG (mM) was not different among FAs. However, in IR it was 94% [46, 159] greater for all FAs (p<0.001). In healthy subjects, the gIC50 (mM) for stearate (0.59 [0.17, 2.09]) and palmitate (0.48 [0.14, 1.70]) were greater than for linoleate (0.10 [0.03, 0.34]) and palmitoleate (0.07 [0.02, 0.25]) (p<0.0001). This difference was not present in IR.

It will be recognized from the foregoing that we have determined, in response to a glucose challenge, trafficking of individual FAs is not uniform. In particular, both saturated FAs had lower Rₐ and higher gIC₅₀ in comparison to other FAs. All FAs except stearate had lower Rₐ in IR. Furthermore, only stearate kNEFA was elevated with IR, indicating stearate trafficking can be a key marker of adipocyte dysfunction in IR.

While the invention has been described through specific embodiments, routine modifications will be apparent to those skilled in the art and such modifications are intended to be within the scope of the present invention.

1. A method for determining insulin resistance and/or adipocyte dysfunction comprising analyzing a sample from an individual to whom a glucose test is administered for a non-esterified fatty acid (NEFA) trafficking parameter comprising a fractional rate of NEFA appearance (Rₐ), a rate of NEFA disappearance (kNEFA), a threshold for glucose-induced suppression (tG), and IC₅₀ for glucose inhibition of NEFA production (gIC₅₀), wherein the at least one NEFA comprises stearate;
and wherein if the Rₐ for stearate is approximately the same as a suitable control, and/or if the kNEFA for stearate is higher than a suitable control, it is indicative that the individual from whom the sample was taken has IR and/or adipocyte dysfunction.

2. The method of claim 1, wherein in addition to stearate the sample is analyzed for the trafficking parameters for one or more of myristic, palmitic, palmitoleic, oleic, and linoleic acids.

3. The method of claim 1, wherein the control for the Rₐ for stearate and/or the control for the kNEFA for stearate is an Rₐ or a kNEFA, respectively, for at least one of myristic, palmitic, palmitoleic, oleic, and linoleic acids.

4. The method of claim 1, wherein the Rₐ for stearate is approximately the same as the suitable control, and/or the kNEFA for stearate is higher than the suitable control, and wherein the individual is selected as a candidate for an exercise program, a change in diet, and/or for a pharmaceutical agent therapy, and/or the individual is diagnosed as having the IR and/or the adipocyte dysfunction.

5. The method of claim 4, wherein the individual is recommended to begin the exercise program, the change in diet, and/or the pharmaceutical agent therapy.

6. The method of claim 5, wherein the individual is prescribed the pharmaceutical agent therapy.

7. The method of claim 6, wherein the pharmaceutical agent comprises a PPAR agonist.

8. The method of claim 7, wherein the PPAR agonist is administered to the individual.

9. (canceled)

10. A method for determining insulin resistance (IR) and/or adipocyte dysfunction comprising analyzing at least a first sample from an individual to whom a glucose challenge is administered for a non-esterified fatty acid (NEFA) trafficking parameter comprising a fractional rate of NEFA appearance (Rₐ), a rate of NEFA disappearance (kNEFA), or a threshold for glucose-induced suppression (tG), or IC₅₀, or a combination thereof, wherein a difference in one or more of the trafficking parameters relative to a control is indicative that the individual has the IR and/or the adipocyte dysfunction.

11. The method of claim 10, wherein the control comprises NEFA trafficking parameters from a sample from the individual that is obtained before the glucose challenge and the first sample is obtained from the individual subsequent to the glucose challenge.

12. The method of claim 11 wherein the first sample is obtained within a period of between 30-90 minutes after the glucose challenge.

13. The method of claim 10 wherein the analyzing comprises determining a ratio of one or more of the trafficking parameters for the control and the first sample.

14. The method of claim 8, wherein the PPAR agonist is administered to the individual in a series of doses over time, and wherein a test is re-administered to assess the efficacy of the PPAR agonist therapy.
